SEQUENCE LISTING (8 sequences)

SEQ ID NO: 1 - DNA, 22 bp, Artificial sequence
  Single strand DNA oligonucleotide
atcagcgtaaatggggatttgg

SEQ ID NO: 2 - DNA, 21 bp, Artificial sequence
  Single strand DNA oligonucleotide
gtctgcggtgatttcatcgaa

SEQ ID NO: 3 - DNA, 21 bp, Artificial sequence
  Single strand DNA oligonucleotide
atggatggacggtaacgggaa

SEQ ID NO: 4 - DNA, 23 bp, Artificial sequence
  Single strand DNA oligonucleotide
cccgatacaaccagtctactaca

SEQ ID NO: 5 - DNA, 22 bp, Artificial sequence
  Single strand DNA oligonucleotide
gctgagaagtcacgttctgaca

SEQ ID NO: 6 - DNA, 22 bp, Artificial sequence
  Single strand DNA oligonucleotide
catctctgtgtcaaccatggta

SEQ ID NO: 7 - DNA, 20 bp, Artificial sequence
  Single strand DNA oligonucleotide
tcgctgatgcactgcctatg

SEQ ID NO: 8 - DNA, 21 bp, Artificial sequence
  Single strand DNA oligonucleotide
gagaggtccacagagctgatt

(19) United States
(12) Patent Application Publication
(10) Pub. No.: US 2017/0298361 A1
(43) Pub. Date: Oct. 19, 2017

(54) USE OF AGENTS FOR TREATING FAT-RELATED DISORDERS

(71) Applicant: Yeda Research and Development Co. Ltd., Rehovot (IL)

(72) Inventors: Yosef SHAUL, Rehovot (IL); Rom David KESHET, Rehovot (IL); Nina REUVEN, Rehovot (IL)

(21) Appl. No.: 15/516,405

(22) PCT Filed: Oct. 1, 2015
(86) PCT No.: PCT/IL2015/050983

§ 371 Date: Apr. 2, 2017

(30) Foreign Application Priority Data
Oct. 2, 2014 (IL) 234962

(51) Int. Cl. C12N 15/113; A61K 31/506; A61K 31/553

(52) U.S. Cl. CPC C12N 15/1137; A61K 31/553; A61K 31/506; C12N 2310/531; C12N 2310/14

(57) ABSTRACT

Use of agents that down-regulates an amount and/or activity of Abelson murine leukemia viral oncogene homolog 1 (c-Abl) or checkpoint kinase 1 (Chk1) in the production of a medicament for treating obesity are disclosed.

### FIELD AND BACKGROUND OF THE INVENTION

The present invention, in some embodiments thereof, relates to methods of treating fat-related disorders, such as obesity.

Obesity, a multifactoral disorder, is a major health problem in developed countries, and is rapidly becoming a significant health problem in developing countries. In the United States, obesity is reaching epidemic proportions. In the adult population, one out of three adults is obese. Excessive adipose tissue is in its strong association with a number of chronic diseases including hyperlipidaemia, high blood pressure, carbohydrate intolerance and diabetes and coronary atherosclerotic heart disease. The precise association between obesity and each of the above chronic diseases is poorly understood, but solid epidemiological data support the role of fat mass. The number of adipocytes in a given fat mass increases in obesity and depends on both hypertrophy of preexisting adipocytes and hyperplasia, the formation of new adipocytes (adipogenesis).

Adipogenesis is a highly regulated process by which preadipocytes mature into adipocytes, and it is one of the most intensively studied models of cellular differentiation. Peroxisome proliferator-activator receptor gamma (PPARγ) is a transcription factor and a pivotal regulator of adipocyte differentiation. It contains an N-terminal transactivation domain (AF1), a DNA binding domain (DBD) and a C-terminal ligand-binding domain (LBD, which harbors the ligand-dependent transactivation domain AF2). The AF1 domain of PPARγ2 is involved in transactivation and recognition of the protein by transcriptional co-activator, and can also influence ligand binding affinity of the LBD. PPARγ dimerizes with retinoid X receptor α (RXRα), and then binds to peroxisome proliferator response elements (PPREs) within the promoter of its targeted genes. PPARγ interacts with a wide spectrum of natural lipophilic ligands and may also exhibit high affinity to synthetic ligands, like the thiazolidinediones. Alternative splicing and differential promoter usage result in two PPARγ isoforms, a ubiquitously expressed PPARγ1 and an adipose restricted PPARγ2, with the latter harboring a 30-residue extension at its N-terminus. Several post-translational modifications of PPARγ are known—most of which are inhibitory, for example sumoylation and phosphorylation by ERK. Interestingly, a proline residue in the PPARγ2 N-terminus is commonly mutated to alanine in the general population. It was found that the P12A substitution in PPARγ2 is associated with decreased receptor activity, lower body mass index and improved insulin sensitivity. Recently it was found that the effect of this mutation on PPARγ2 activity is the result of a reduced interaction of PPARγ2 with some of its coactivators.

The non-receptor tyrosine kinase c-Abl is known to regulate several cellular processes and to participate in cell fate decisions like induction of apoptosis or cell cycle arrest. However, c-Abl is involved in differentiation as well. Interestingly, c-Abl was found to be highly expressed in human adipocytes (O'Neill A J, et al. (1997) The Journal of pathology 183(3):325-329), and to be activated upon insulin stimulation, where it participates in regulation of metabolic downstream signals (Frasca F, et al. (2007) The Journal of biological chemistry 282(36):26077-26088). Furthermore, a recent proteomic analysis placed c-Abl as a potential key regulator of adipogenesis (Wilson B, Liotta L A, & Petricoiniii E (2013) Molecular & cellular proteomics: MCP 12(9):2522-2535).

The c-Abl inhibitor STI-571 (Imatinib) was shown to promote adipocytic differentiation of mesenchymal stromal cells (Fitter S, et al. (2008) Blood 111(5):2538-2547).

STI-571 treatment in CML patients was associated with fat accumulation and improved insulin sensitivity (Fitter S, et al. (2010) The Journal of clinical endocrinology and metabolism 95(8):3763-3767).

Additional background art includes US Patent Application No. 20060116403.

### SUMMARY OF THE INVENTION

According to an aspect of some embodiments of the present invention there is provided a method of treating obesity in a subject in need thereof, comprising administering to the subject a therapeutically effective amount of an agent which directly down-regulates an amount and/or activity of Abelson murine leukemia viral oncogene homolog 1 (c-Abl) and/or checkpoint kinase 1 (Chk1), thereby treating obesity in the subject.

According to an aspect of some embodiments of the present invention there is provided a method of reducing fat content or body weight in a subject, comprising administering to the subject an effective amount of an agent which down-regulates an amount and/or activity of Abelson murine leukemia viral oncogene homolog 1 (c-Abl) and/or checkpoint kinase 1 (Chk1), thereby reducing fat content or body weight in the subject.

According to an aspect of some embodiments of the present invention there is provided a method of reducing adipocyte differentiation in a subject, comprising administering to the subject an effective amount of an agent which down-regulates an amount and/or activity of Abelson murine leukemia viral oncogene homolog 1 (c-Abl) and/or checkpoint kinase 1 (Chk1), thereby reducing adipocyte differentiation in the subject.

According to an aspect of some embodiments of the present invention there is provided a method of increasing the amount of fat in a subject, comprising administering to the subject an effective amount of an agent which up-regulates an amount and/or activity of Abelson murine leukemia viral oncogene homolog 1 (c-Abl) or checkpoint kinase 1 (Chk1), thereby increasing the amount of fat in the subject.

According to an aspect of some embodiments of the present invention there is provided a use of an agent that down-regulates an amount and/or activity of Abelson murine leukemia viral oncogene homolog 1 (c-Abl) or checkpoint kinase 1 (Chk1) in the production of a medicament for treating obesity.

According to an aspect of some embodiments of the present invention there is provided a use of an agent that down-regulates an amount and/or activity of Abelson murine leukemia viral oncogene homolog 1 (c-Abl) or checkpoint kinase 1 (Chk1) in the production of a medicament for reducing the amount of fat in a subject.

According to an aspect of some embodiments of the present invention there is provided a use of an agent that up-regulates an amount and/or activity of Abelson murine leukemia viral oncogene homolog 1 (c-Abl) or checkpoint kinase 1 (Chk1) in the production of a medicament for increasing the amount of fat in a subject.

According to an aspect of some embodiments of the present invention there is provided a use of an agent that down-regulates an amount and/or activity of Abelson murine leukemia viral oncogene homolog 1 (c-Abl) or checkpoint kinase 1 (Chk1) in the production of a medicament for reducing the amount adipocyte differentiation in a subject.

According to some embodiments of the invention, the subject has the amino acid proline at position 12 of Peroxisome proliferator-activator receptor gamma (PPARγ2).

According to some embodiments of the invention, the agent is a small molecule inhibitor.

According to some embodiments of the invention, the agent is a polynucleotide agent directed against said c-Abl or said Chk1.

According to some embodiments of the invention, the agent is selected from the group consisting of imatinib (STI-571), nilotinib, bosutinib, INNO-406, MK-0457 and PD173955.

According to some embodiments of the invention, the agent is UCN-01 or LY2606368.

According to some embodiments of the invention, the subject does not have cancer.

According to some embodiments of the invention, the subject does not have diabetes.

According to some embodiments of the invention, the subject does not have pathological lipid levels.

According to some embodiments of the invention, the subject does not have atherosclerosis.

According to some embodiments of the invention, the subject does not have pathological cholesterol levels.

According to some embodiments of the invention, the therapeutically effective amount reduces 5% of body weight of the subject within one year of administration.

According to some embodiments of the invention, the subject has been treated for at least one month to correct for elevated lipid levels.

According to some embodiments of the invention, the agent is not co-administered with an anti-cancer agent.

According to some embodiments of the invention, the agent is not co-administered with an agent used to treat atherosclerosis.

According to some embodiments of the invention, the agent is a small molecule inhibitor.

According to some embodiments of the invention, the agent is a polynucleotide agent directed against said c-Abl or said Chk1.

According to some embodiments of the invention, the agent is selected from the group consisting of imatinib (STI-571), nilotinib, Bosutinib, bosutinib, INNO-406, MK-0457 and PD173955.

According to some embodiments of the invention, the agent is UCN-01.

According to some embodiments of the invention, the agent is not co-administered with an anti-cancer agent.

According to some embodiments of the invention, the agent is not co-administered with agent used to treat atherosclerosis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E illustrate that c-Abl is required for adipocyte differentiation. (A) c-Abl activity increases during adipogenesis. 3T3-L1 adipoblasts were induced to differentiate with medium containing 5 μg/ml Insulin, 1 μM Dexamethasone and 0.5 mM IBMX. Cells were lysed at the indicated times and protein levels were analyzed by Western blotting. The proteasomal subunit PSMA4 served as a loading control. (B) STI-571 inhibits adipocyte differentiation. 3T3-L1 cells were induced to differentiate as described in (A), with STI-571 at the indicated concentrations or DMSO being added to the medium on days 0-5. Cells were stained with oil red O and photographed at day 8. (C) c-Abl is required in adipocyte differentiation. 3T3-L1 expressing shRNA against either GFP or c-Abl were induced to differentiate and stained with oil red 0 at day 10. (D) c-Abl is required for expression of PPARγ and its target genes during adipogenesis. 3T3-L1 cells expressing shRNA against either GFP or c-Abl were induced to differentiate as in (A). Cells were lysed at the indicated times and protein levels were analyzed by Western blotting. (E) c-Abl is required for transcription of PPARγ pro-adipogenic target genes but not for transcription of PPARγ itself. At day 4 of differentiation, RNA from 3T3-L1 cells was analyzed for mRNA expression of the indicated genes by quantitative PCR.

FIGS. 2A-C illustrate that c-Abl prolongs PPARγ2 half-life. (A) Proteasomal inhibition in mid-differentiation results in PPARγ2 accumulation. 3T3-L1 cells expressing shRNA to GFP (control) or c-Abl were induced to differentiate using insulin, dexamethasone and IBMX. On day 5, cells were treated with 25 μg MG-132 and lysed after 5 h. Protein levels were analyzed by immunoblotting. (B) c-Abl stabilizes PPARγ2. HEK293 cells were transfected with plasmids expressing HA-PPARγ2 with or without active c-Abl. Twenty-four hours after transfection, cells were treated with 20 μg/ml cycloheximide for different time points. Equal amounts of total protein lysates were subjected to western blotting analysis with the indicated antibodies. (C) c-Abl inhibits PPARγ2 poly-ubiquitination. HEK293 cells were transfected as indicated. Cell extracts were immunoprecipitated using Flag beads and immunoblotted with anti-HA to visualize poly-ubiquitination. The ratio between immunoprecipitated poly-ubiquitinated PPARγ2 ladder and PPARγ2 protein level from 3 independent experiments was quantified (*** −p<0.005). Immunoblot data from representative experiment shown in FIG. 9.

FIGS. 3A-E illustrate that c-Abl binds and phosphorylates PPARγ2. (A) c-Abl binds PPARγ2. HEK293 cells were transfected with the indicated plasmids and analyzed by co-immunoprecipitation. (B) PXXP motifs within PPARγ—arrows denote transcription start sites for PPARγ1 and PPARγ2. The three PXXP motifs in the AF1 domain are highlighted. AF—activation function domains; DBD—DNA binding domain; LBD—ligand binding domain. Image modified from Tontonoz and Spiegelman, Annu. Rev. Biochem. 2008. (C) The N-terminal PXXP motif of PPARγ2 is required for interaction with c-Abl. HEK293 cells were transfected with the indicated plasmids and analyzed by immunoprecipitation with anti-flag beads. (D) c-Abl phosphorylates PPARγ2 but not PPARγ1. HEK293 cells were transfected with the indicated plasmids and analyzed by immunoprecipitation using anti-HA beads. Phosphorylation was detected by probing with an anti-phosphotyrosine antibody (PY20). (E) The mobility shift in the PPARγ2 band represents a phosphorylated form of PPARγ2. HEK293 cells were transfected with the indicated plasmids. Cell lysates were incubated in 37° C. for 30 minutes in the presence or absence of 150 U/ml Alkaline Phosphatase (CIP) and were then analyzed by immunoprecipitation using anti-Flag beads.

FIGS. 4A-E illustrate that two tyrosine residues in the AF1 domain of PPARγ2 are the phosphorylation targets of c-Abl. (A) Tyr78 and Tyr102 are c-Abl phosphorylation sites in PPARγ2. HEK293 cells were transfected with the indicated plasmids and analyzed by immunoprecipitation. Phosphorylation was detected by probing with an anti-phosphotyrosine antibody (PY20). (B) Differentiation induction enhances phosphorylation of Tyr78 and Tyr102 by endogenous c-Abl. NIH-3T3 fibroblasts were infected with control pBabe retroviral vector or with pBabe carrying the indicated PPARγ2 constructs. Cells were cultured to confluence and then induced to differentiate with medium containing 5 μg/ml Insulin, 1 μM Dexamethasone, 0.5 mM IBMX and 1 μM Rosiglitazone. On day 5 of differentiation, cells were lysed and analyzed by immunoprecipitation. (C) Phosphomimetic mutations on tyrosine residues 78 and 102 result in PPARγ2 stabilization. HEK293 cells were transfected with plasmids expressing the different Flag-PPARγ2constructs. Twenty-four hours after transfection, cells were treated with 500 μM cycloheximide for different time points. Equal amounts of total protein lysates were subjected to western blotting analysis with the indicated antibodies. (D) Phosphomimetic mutations on tyrosine residues 78 and 102 inhibit PPARγ2 poly-ubiquitination. HEK293 cells were transfected as indicated. Cell extracts were immunoprecipitated using anti-Flag beads and immunoblotted with anti-HA to visualize poly-ubiquitination. (E) Quantification of the ratio between immunoprecipitated poly-ubiquitinated PPARγ2 ladder and PPARγ2 protein level from 3 independent experiments (*−p<0.05).

FIGS. 5A-D illustrate that c-Abl potentiates PPARγ2 activity. (A) PPARγ2 transcriptional activity is enhanced by c-Abl. The designated plasmids were co-transfected with a PPRE3-Luciferase reporter plasmid into HEK293 cells. After 24 h, cells were lysed and luciferase activity was measured and compared to vector control transfected cells. (B) PPARγ2 transcriptional activity is enhanced by phosphomimetic mutations on tyrosine residues 78 and 102. The designated plasmids were co-transfected with a PPRE3-Luciferase reporter plasmid into HEK293 cells. RXRα was cotransfected with all PPARγ2 constructs and Rosiglitazone (1 μM) was added 6 h after transfection. After 24 h, cells were analysed as in (A). (C) Tyr78 and Tyr102 phosphorylation promotes adipocyte differentiation. NIH-3T3 fibroblasts were infected with control pBabe retroviral vector or with pBabe carrying the indicated PPARγ2 constructs. Cells were cultured to confluence and then induced to differentiate with medium containing Rosiglitazone 1 μM . On day 10 of differentiation, cells were fixed and stained with oil red. (D) Tyr78 and Tyr102 phosphorylation promotes PPARγ target gene induction during adipogenesis. NIH-3T3 fibroblasts were transduced with the indicated PPARγ2 constructs and were induced to differentiate as in (C). At day 4 of differentiation, RNA from NIH-3T3 cells expressing the indicated PPARγ constructs was analyzed for mRNA expression of the indicated genes by quantitative PCR.

FIGS. 6A-C illustrate that c-Abl binds PPARγ through a genetic polymorphism site (A) The P12A mutation of PPARγ2 interferes with c-Abl potential to bind and phosphorylate PPARγ2. HEK293 cells were transfected with the indicated plasmids and analyzed by immunoprecipitation using anti-Flag beads. Phosphorylation was detected by probing with an anti-phosphotyrosine antibody (PY20). (B) The P12A mutation of PPARγ2 inhibits stabilization by c-Abl. HEK293 cells were transfected with plasmids expressing P12A mutant PPARγ2 with or without active c-Abl. After 24 h, cells were treated with 500 μM cycloheximide for different time points. Equal amounts of total protein lysates were subjected to western blotting analysis with the indicated antibodies. (C) Phosphorylation by c-Abl favors binding of PPARγ2 to PGC-1α. HEK293 cells were transfected with the indicated plasmids and analyzed by co-immunoprecipitation.

FIG. 7 is a model portraying regulation of adipogenesis by c-Abl. Under conditions by which c-Abl is not active, PPARγ2 is unstable. Normal adipogenesis is accompanied by c-Abl activation, resulting in PPARγ2 phosphorylation at tyrosine residues 78 and 102, and its subsequent stabilization and binding to transcriptional coactivators. DBD—DNA binding domain; LBD—ligand binding domain; Ub—ubiquitin; P—phosphorylated tyrosine residues; L—PPARγ2 ligand (in red) or RXRα ligand (in grey).

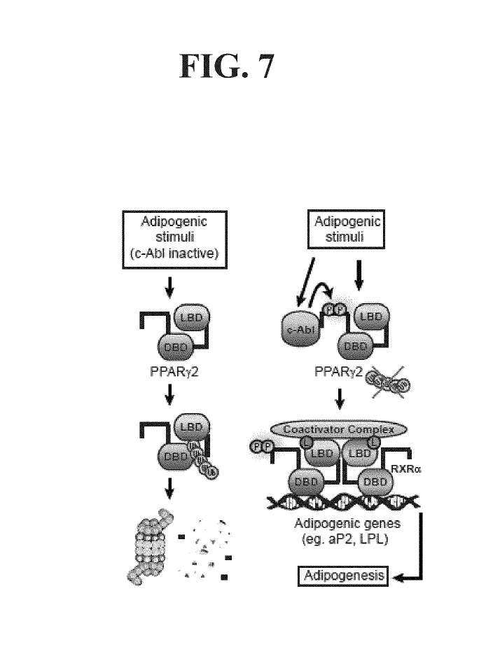

FIG. 8 shows c-Abl protein levels in 3T3-L1 cells infected with a lentiviral plasmid containing shRNA against either GFP or c-Abl.

FIG. 9 illustrates that c-Abl inhibits PPARγ2 poly-ubiquitination. HEK293 cells were transfected as indicated. Cell extracts were immunoprecipitated using Flag beads and immunoblotted with anti-HA to visualize poly-ubiquitination.

FIGS. 10A-B (A) c-Abl binds PPARγ2. HEK293 cells were transfected with the indicated plasmids and analyzed by co-immunoprecipitation. (B) c-Abl phosphorylates PPARγ2. HEK293 cells were transfected with the indicated plasmids and analyzed by immunoprecipitation using anti-flag beads. Phosphorylation was detected by probing with a phosphotyrosine antibody (PY20).

FIGS. 11A-B (A) Tyr102 is a c-Abl phosphorylation site in PPARγ2. HEK293 cells were transfected with the indicated plasmids and analyzed by immunoprecipitation. Phosphorylation was detected by probing with a phosphotyrosine antibody (PY20). (B) Blocking phosphorylation of Tyr78 and Tyr102 prevents total PPARγ2 phosphorylation by c-Abl. HEK293 cells were transfected with the indicated plasmids and analyzed by immunoprecipitation. Phosphorylation was detected by probing with a phosphotyrosine antibody (PY20).

FIGS. 12A-D (A) c-Abl potentiation of PPARγ2 activity is kinase-dependent. The designated plasmids were co-transfected with a PPRE3-Luciferase reporter plasmid into HEK293 cells. After 24 h, cells were lysed and luciferase activity was measured and compared to vector control transfected cells. (B) The 2YE mutation enhances PPARγ2 activity over time. The designated plasmids were cotransfected with a RXRα and PPRE3-Luciferase reporter plasmid into HEK293 cells and Rosiglitazone (1 μM) was added 6 h after transfection. Luciferase activity was recorded over 67 h using a real time bioluminescence detector. (C) Tyr78 phosphorylation promotes adipocyte differentiation. NIH-3T3 fibroblasts were infected with control pBabe retroviral vector or with pBabe carrying the indicated PPARγ2 constructs. Cells were cultured to confluence and then differentiated with medium containing Rosiglitazone 1 μM. On day 10 of differentiation, microscopic images were taken (lower panels) and then cells were fixated, stained with oil red and photographed (upper panels). (D) Tyr102 phosphorylation promotes adipocyte differentiation. NIH-3T3 fibroblasts were infected with control pBabe retroviral vector or with pBabe carrying the indicated PPARγ2 constructs. Cells were cultured to confluence and then induced to differentiate with medium containing 5 μg/ml Insulin, 1 μM Dexamethasone and 0.5 mM IBMX. On day 10 of differentiation, cells were fixed and stained with oil red.

FIG. 13 c-Abl stabilizes PPARγ2. HEK293 cells were transfected with plasmids expressing PPARγ2 with or without active c-Abl. Twenty-four hours after transfection, cells were treated with 500 μM cycloheximide for different time points. Equal amounts of total protein lysates were subjected to Western blotting analysis with the indicated antibodies.

FIG. 14 The expression of Chk1 and phospho-Chk 1 in 3T3-L1 differentiation cells. 3T3-L1 preadipocytes were differentiated with medium containing Insulin 5 μg/ml, Dexamethasone 1 μM, and IBMX 0.5 mM. Cells were lysed at the indicated time points, protein levels were analyzed by Western blotting. The elevation of Chk1 on day 1 was most probably due to a mitotic clonal expansion of confluent preadipocytes, following hormonal stimulation. Chk1 phosphorylation in non-cycling confluent preadipocytes, prior to induction towards differentiation, may imply that Chk1 is activated in confluent pre-adipocytes. (Ex-exponentially growing cells; Co-undifferentiated confluent cells, D1-D7—days after differentiation).

FIG. 15 are photographs illustrating differentiation of 3T3-L1 preadipocytes with or without Chk1 inhibitor, UCN-01.

### DESCRIPTION OF SPECIFIC EMBODIMENTS OF THE INVENTION

The present invention, in some embodiments thereof, relates to methods of treating fat-related disorders, such as obesity. The method comprises administering agents that down-regulate components of the DNA damage response (DDR) pathway including Abelson murine leukemia viral oncogene homolog 1 (c-Abl) and/or checkpoint kinase 1 (Chk1).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Adipocytes evolve from pre-adipocyte progenitors by the process of adipogenesis. This extensively studied process is regulated by complex signaling networks, all governed by the adipogenic master regulator PPARγ, a transcription factor of the nuclear receptor superfamily. PPARγ is also involved in metabolic homeostasis, in pathological states such as obesity and diabetes.

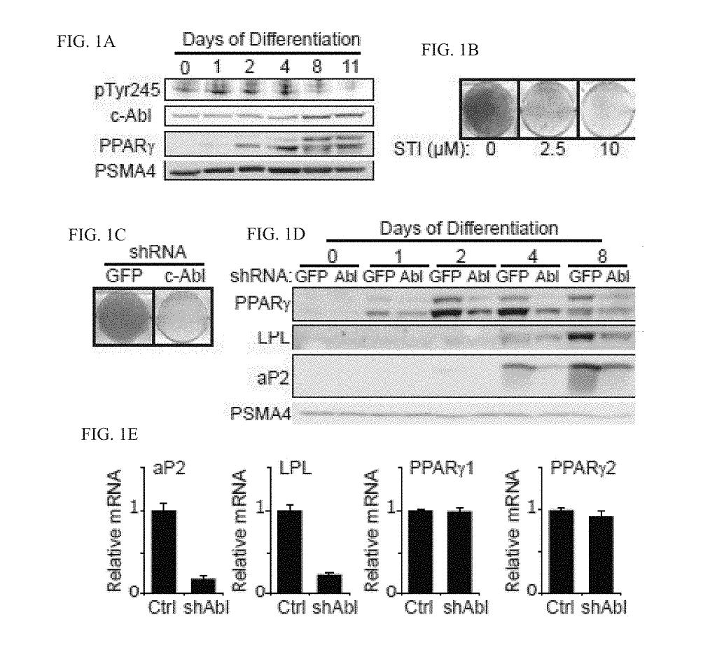

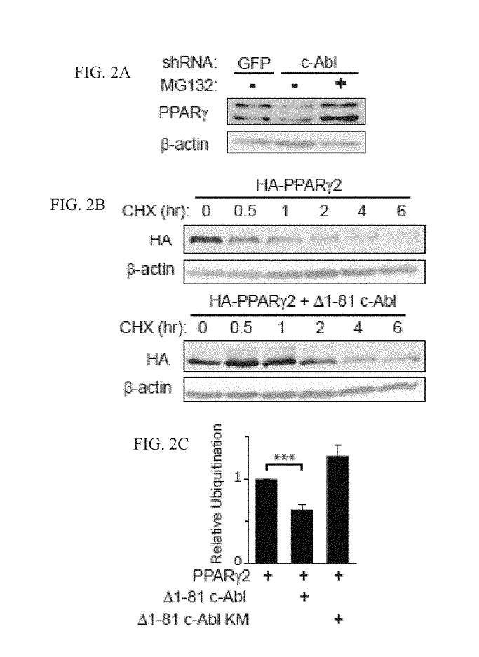

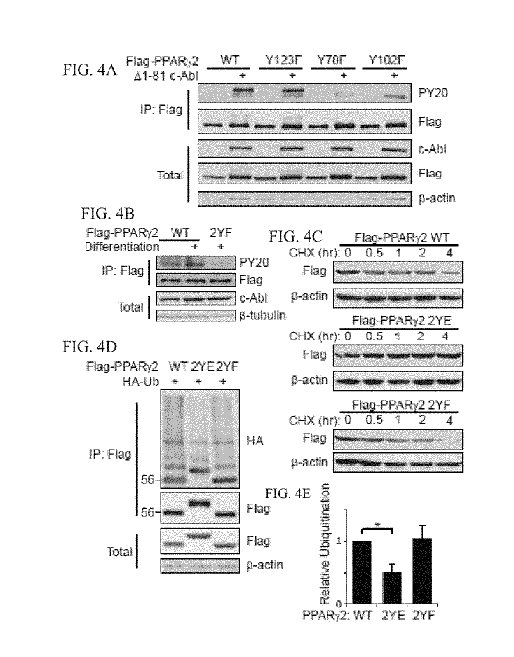

The present inventors have found that c-Abl was activated during the early phase of mouse 3T3-L1 pre-adipocytes differentiation. Moreover, c-Abl activity was essential and its inhibition blocked differentiation into mature adipocytes (FIGS. 1A-E). The present inventors showed that c-Abl directly controlled the expression and activity of the master adipogenic regulator PPARγ2. PPARγ2 physically associated with c-Abl and underwent phosphorylation on two tyrosine residues within its regulatory AF-1 domain (FIGS. 4A-E). The present inventors demonstrated that this process positively regulates PPARγ2 stability and brings about adipogenesis (FIGS. 2A-C and 5A-D).

A common genetic polymorphism of PPARy, encoding the pro12ala (P12A) variant, is associated with a slim phenotype. Remarkably, c-Abl binding to PPARγ2 requires the P12 residue that has a phenotypically well-studied common human genetic P12A polymorphism. Importantly, the reduced binding affinity of the P12A variant to c-Abl provides an explanation for the phenotype of this genetic variant. The present findings establish a critical role for c-Abl in adipocyte differentiation and explain the behavior of the known P12A polymorphism. Thus, the present inventors propose that inhibiting c-Abl might mimic the P12A polymorphic manifestation and as such be of use in treating obesity and other fat-related disorders.

Thus, according to one aspect of the present invention there is provided a method of reducing fat content or body weight (or reducing adipocyte differentiation) in a subject, comprising administering to the subject an effective amount of an agent which directly down-regulates an amount and/or activity of Abelson murine leukemia viral oncogene homolog 1 (c-Abl) and/or checkpoint kinase 1 (Chk1), thereby reducing fat content or body weight in the subject.

As used herein the phrase “fat content” refers to the percentage of body weight attributed to fat tissue.

According to one embodiment, the agents of the present invention act by decreasing adipogenesis—i.e. decreasing the number of adipocytes, also referred to herein as decreasing hyperplasia. Hyperplasia results from the formation of new adipocytes from precursor cells in adipose tissue. Typically hyperplasia involves the proliferation of preadipocytes and their differentiation into adipocytes.

Additionally, or alternatively, the agents of the present invention act by decreasing adipocyte hypertrophy (the increase in size of a pre-existing adipocyte as a result of excess triglyceride accumulation).

c-ABL kinase (mRNA: NM_005157; protein: NP_005148) is a non-receptor tyrosine kinase (EC No. 2.7.10.2) involved in cell signal transduction, the gene encoding same being on chromosome 9; 133.59-133.76 Mb. This ubiquitously expressed kinase, (upon activation by upstream signaling factors including growth factors, oxidative stress, integrin stimulation, and ionizing radiation), localizes to the cell plasma membrane, the cell nucleus, and other cellular compartments including the actin cytoskeleton. There are two normal isoforms of Abl kinase: ABL-1A and ABL-1B. The N-terminal half of c-ABL kinase is important for autoinhibition of the kinase domain catalytic activity. The N-terminal myristoyl amino acid residue of ABL-1B has been shown to intramolecularly occupy a hydrophobic pocket formed from alpha-helices in the C-lobe of the kinase domain. Such intramolecular binding induces a novel binding area for intramolecular docking of the SH2 domain and the SH3 domain onto the kinase domain, thereby distorting and inhibiting the catalytic activity of the kinase. Thus, an intricate intramolecular negative regulation of the kinase activity is brought about by these N-terminal regions of c-ABL kinase.

Checkpoint kinase 1 (Chk1; mRNA: NM_001114121; protein: NP_001107593) is a Serine/threonine-specific protein kinase (EC No. 2.7.11.1) that in humans, is encoded by the CHEK1 gene. Chk1 coordinates the DNA damage response (DDR) and cell cycle checkpoint response. Activation of Chk1 results in the initiation of cell cycle checkpoints, cell cycle arrest, DNA repair and cell death to prevent damaged cells from progressing through the cell cycle.

The present invention contemplates use of agents that directly down-regulate an amount and/or activity of c-ABL or Chk1.

Examples of such agents include those that bind directly to c-ABL or Chk1, blocking or down-regulating its activity. Other agents that down-regulate activity of c-ABL or Chk1 are those that bind to a direct target of c-ABL or Chk1, preventing the interaction of c-ABL or Chk1 with its target. Other methods to down-regulate c-Abl or Chk1 include, but are not limited to, knockout technology, antisense technology, triple helix technology, targeted mutation, gene therapy or regulation by-agents acting on transcription, as further described herein below.

Contemplated agents include proteins, nucleic acids, carbohydrates, antibodies, or any molecules including ligands which can bind to c-Abl either directly or indirectly to induce an effect on c-Abl. Other “antagonists” or “inhibitors” include a range of rationally designed, synthetic inhibitors, generally based on direct inhibitors of c-Abl or Chk1.

According to one embodiment, the c-Abl inhibitor is imatinib or an equivalent including for example imatinib mesylate, N-desmethyl imatinib. An equivalent of imatinib as used herein means a compound which behaves in a similar manner to imatinib so as not to be able to distinguish between the two compounds. The equivalent should preferably target c-Abl at the same sites as imatinib and therefore induce the same outcome as imatinib. The inhibitor imatinib is a 2-phenyl aminopyridine derivative that has recently been introduced for the treatment of human chronic myeloidleukemia (CML) in which the c-Abl is constitutively activated. It has inhibitory effects on the growth of cancer cells in which c-Abl is activated but little effect on non-transformed cells.

Additional agents capable of down-regulating cAbl include but are not limited to nilotinib, dasatinib (anhydrous or monohydrate), bosutinib and ponatinib.

According to another embodiment the c-Abl inhibitor is herbimycinA, 1-Napthyl PP1, chlorogenic acid, AP 24534, Bcr-abl Inhibitor, Bcr-abl Inhibitor II, PP121 AT9283, DCC-2036 or imatinib.

According to another embodiment, the c-Abl inhibitor is selected from the group consisting of imatinib, nilotinib, bosutinib, INNO-406, MK-0457 and PD173955.

Exemplary Checkpoint kinase 1 inhibitors contemplated by the present inventor include, but are not limited to UCN-01 and LY2603618.

Additional agents contemplated by the present inventors include CHIR-124, debromohymenialdisine, SB 218078, Chk2 inhibitor, AZD7762, SCH 900776, TCS 2312, PD 407824, PF 477736 and CHIR-124.

Additional examples of Checkpoint kinase 1 inhibitors are disclosed in Janetka et al., Expert Opin Ther Pat. 2009 February; 19(2):165-97, the contents of the patents disclosed therein being incorporated herein by reference.

As mentioned, downregulation of c-Abl or Chk1 can be effected on the genomic and/or the transcript level using a variety of molecules which interfere with transcription and/or translation [e.g., RNA silencing agents (e.g., antisense, siRNA, shRNA, micro-RNA), Ribozyme and DNAzyme], or on the protein level using e.g., antagonists, enzymes that cleave the polypeptide and the like.

Following is a list of agents capable of downregulating expression level and/or activity of c-Abl or Chk1.

One example, of an agent capable of downregulating c-Abl or Chk1 is an antibody or antibody fragment capable of specifically binding c-Abl or Chk1.

Preferably, the antibody specifically binds at least one epitope of c-Abl or Chk1. As used herein, the term “epitope” refers to any antigenic determinant on an antigen to which the paratope of an antibody binds.

Downregulation of c-Abl or Chk1 can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to an embodiment of the invention, the RNA silencing agent is specific to the target RNA (e.g., c-Abl or Chk1) and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the invention according to some embodiments thereof contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi: 10.1089/154545703322617069.

The invention according to some embodiments thereof also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 2lmers with a central 19 bp duplex region and symmetric 2-base 3′-overhangs on the termini, although it has been recently described that chemically synthesized RNA duplexes of 25-30 base length can have as much as a 100-fold increase in potency compared with 2lmers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the c-Abl or Chk1mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level.

Second, potential target sites are compared to an appropriate genomic database (e.g., human, mouse, rat etc.) using any sequence alignment software, such as the BLAST software available from the NCBI server. Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that the RNA silencing agent of some embodiments of the invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, plsl, TAT(48-60), pVEC, MTS, and MAP.

According to another embodiment the RNA silencing agent may be a miRNA or miRNA mimic.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms and have been shown to play a role in development, homeostasis, and disease etiology.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Another agent capable of downregulating c-Abl or Chk1 is a DNAzyme molecule capable of specifically cleaving an mRNA transcript or DNA sequence of c-Abl or Chk1. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab 1 oncogenes were successful in inhibiting the oncogenes expression in leukemia cells, and lessening relapse rates in autologous bone marrow transplant in cases of CML and ALL.

Downregulation of c-Abl or Chk1 can also be effected by using an antisense polynucleotide capable of specifically hybridizing with an mRNA transcript encoding the c-Abl or Chk1.

Design of antisense molecules which can be used to efficiently downregulate a c-Abl or Chk1 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374 - 1375 (1998)].

Another agent capable of downregulating c-Abl or Chk1 is a ribozyme molecule capable of specifically cleaving an mRNA transcript encoding c-Abl or Chk1. An additional method of regulating the expression of c-Abl or Chk1 gene in cells is via triplex forming oligonuclotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science,1989; 245:725-730; Moser, H. E., et al., Science,1987; 238:645-630; Beal, P. A., et al, Science,1992; 251:1360-1363; Cooney, M., et al., Science,1988; 241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the c-Abl or Chk1 regulatory region a triplex forming sequence may be devised. Triplex-forming oligonucleotides preferably are at least 15, more preferably 25, still more preferably 30 or more nucleotides in length, up to 50 or 100 bp.

The subjects who are treated with the agents described herein are typically mammalian (e.g. humans).

According to a particular embodiment, the subject being treated does not have a genetic polymorphism in the gene encoding Peroxisome proliferator-activator receptor gamma (PPARγ2). Thus, preferably position 12 of the amino acid sequence of PPARγ2 of the subject is proline (and not alanine).

The subjects who are treated preferably do not have cancer (e.g. Chronic myelogenous leukemia; CML).

The subjects who are treated preferably do not have Diabetes.

The subjects who are treated preferably do not have atherosclerosis, or show symptoms of atherosclerosis.

The subjects who are treated preferably do not have pathological lipid levels (i.e. similar lipid levels as those people having a metabolic lipid disorder). It will be appreciated that the subject may have normal (non-pathological lipid levels) because he is essentially healthy or because he has been pre-treated (e.g. for at least one month) with an agent which effectively reduces his lipid levels to normal.

The subjects who are treated preferably do not have pathological cholesterol levels (i.e. similar cholesterol levels as those people having a cholesterol-related disorder). It will be appreciated that the subject may have normal (non-pathological cholesterol levels) because he is essentially healthy or because he has been pre-treated (e.g. for at least one month) with an agent (for example a statin) which effectively reduces his cholesterol levels to normal.

The subjects who are treated preferably do not have pathological blood pressure levels (i.e. similar blood pressure levels as those people having atherosclerosis). It will be appreciated that the subject may have normal (non-pathological blood pressure levels) because he is essentially healthy or because he has been pre-treated (e.g. for at least one month) with an agent which effectively reduces blood pressure.

Thus, the subject may be healthy (show no sign of a disease or disorder) or may have a disease or condition which has been corrected.

According to one embodiment the subject has a body mass index (BMI) of greater than 30. Subjects having BMI between 25 and 30 are considered overweight and in one embodiment, are treated by the agents disclosed herein. The body mass index (BMI) is calculated by dividing an individual's weight in kilograms by the square of their height in meters. BMI does not distinguish fat mass from lean mass and an obese subject typically has excess adipose tissue.

Thus, in one embodiment of the present invention, the subject has a BMI greater than 30. In one embodiment, the subject has a BMI of 25 or over, e.g. 26, 27, 28, 29, 30, 31, 32, 33, 34, 35, 36, 37, 38, 39 or 40 or greater and has no obesity-related co-morbidity. In one embodiment, the patient is morbidly obese and has a BMI of 40 or over. In one embodiment, the subject is obese and/or suffering from complications associated with obesity. In one embodiment, the subject is obese and/or was suffering from complications associated with obesity, which have now been corrected. In one embodiment, the subject has a Body Mass Index (BMI) of over 25, and preferably over 30.

The agents of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the c-ABL inhibitors and Chk1 inhibitors accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the

BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (c-ABL inhibitors or Chk1 inhibitors) effective to prevent, alleviate or ameliorate symptoms of a disorder (e.g., obesity).

According to a particular embodiment, the therapeutically effective amount refers to an amount which reduces BMI compared to baseline or a placebo arm.

According to a particular embodiment, the therapeutically effective amount refers to an amount which reduces body weight compared to baseline or a placebo arm.

According to a particular embodiment, the therapeutically effective amount refers to an amount which reduces waist measurement compared to baseline or a placebo arm

According to a particular embodiment, the therapeutically effective amount refers to an amount which reduces body fat percentage compared to baseline or a placebo arm.

The treatment may be effected for any length of time, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week.

According to one embodiment, the treatment is effective when over the course of 3 months, 4 months, 5 months, 6 months, 7 months, 8 months, 9 months, 10 months, 11 months or a year, the BMI is reduced by at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline or a placebo arm.

According to one embodiment, the treatment is effective when over the course of 3 months, 4 months, 5 months, 6 months, 7 months, 8 months, 9 months, 10 months, 11 months or a year, the body weight is reduced by at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline or a placebo arm.

According to one embodiment, the treatment is effective when over the course of 3 months, 4 months, 5 months, 6 months, 7 months, 8 months, 9 months, 10 months, 11 months or a year, the waist measurement is reduced by at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline or a placebo arm.

According to one embodiment, the treatment is effective when over the course of 3 months, 4 months, 5 months, 6 months, 7 months, 8 months, 9 months, 10 months, 11 months or a year, body fat percentage is reduced by at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline or a placebo arm.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or months or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The agents of the present invention are preferably not co-formulated with additional anti-lipid, anti-cholesterol, anti-atherosclerosis, anti-diabetes or anti-cancer agents.

According to one embodiment, the agents are not co-administered with anti-lipid agents, anti-cholesterol agents, anti-atherosclerosis agents, anti-diabetes agents, or anti-cancer agents.

According to one embodiment, the subjects have not been pre-treated with anti-lipid agents, anti-cholesterol agents, anti-atherosclerosis agents, anti-diabetes agents, or anti-cancer agents.

In one embodiment, the agent is co-administered with a lipid lowering drug such as peroxisome proliferating activating receptor alpha ligands, probucol, and nicotinic acid.

As mentioned hereinabove, the present invention also envisages upregulation of c-ABL or Chk1 activity or expression, since such upregulation is expected to increase total body weight and fat content, a feature which finds utility when increasing body weight and/or fat content is desired.

Upregulation of c-ABL or Chk1 expression levels can be effected by delivering to a subject an exogenous polynucleotide sequence designed and constructed to express at least a functional portion of the c-ABL or Chk1 in the subject.

In order to generate a polynucleotide construct capable of expressing at least a functional portion of c-ABL or Chk1 according to the present invention, a polynucleotide segment encoding c-ABL or Chk1 or a functional portion thereof can be ligated into a commercially available expression vector system suitable for transforming mammalian cells and for directing the expression of c-ABL or Chk1 within the transformed cells. Preferably the construct will further include a suitable promoter.

It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

Suitable mammalian expression vectors for use with the present invention include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives.

Increasing c-ABL or Chk1 expression levels can also be effected by an agent(s) which increases expression of endogenous c-ABL or Chk1.

Subject who are treated with agents which increase the level of c-ABL or Chk1 typically have a BMI which is less than 18.5, less than 17, less than 16 or less than 15.

The subject may have a disease such as hyperthyroidism or anorexia nervosa. Alternatively, the subject may have a disease such as cancer.

Upregulators of c-ABL or Chk1 may be provided per se or may be administered in pharmaceutical compositions as described herein above.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Cells and Cell Culture: The cell lines used were human embryonic kidney cells HEK293 and HEK293T, NIH-3T3 mouse fibroblasts and the mouse pre-adipocyte cells 3T3-L1. Culture conditions, reagents, plasmids, transfections, mRNA analysis, immunoblot and coimmunoprecipitation studies are described below.

Reporter gene assays: HEK293 cells were transfected with plasmids expressing the tested constructs along with a promoter-containing firefly luciferase reporter plasmid. Cell lysates were analyzed as described below.

Statistical analysis: All values presented in graphs represent the average of at least three independent experiments. To estimate distribution of values, standard error was calculated. The two-tailed student t-test was used to verify statistical significance in the difference between relevant values.

Cells and Cell Culture: HEK 293 cells were grown in Dulbecco's modified Eagle's medium (DMEM; GIBCO) supplemented with 8% fetal bovine serum (GIBCO), 100 units/ml penicillin, 100 μg/ml streptomycin and cultured at 37° C. in a humidified incubator with 5% CO2. The 3T3-L1 cells were grown in the same medium except adult bovine serum was used instead of fetal bovine serum. Light microscopy photographs of cells were performed using an Olympus IX70 microscope connected to a DVC camera. Reagents added to culture medium were Cycloheximide and MG132 (Sigma); STI-571, which was kindly provided by Novartis; and Rosiglitazone (Enzo Life Sciences).

Plasmids, transfection and mRNA analysis: pCDNA c-Abl Δ1-81 and pCDNA c-Abl Δ1-81 K290H (kinase-dead) were previously described (Reuven N et al., Cell death and differentiation 20(10):1330-1340). HA tagged PPARγ1 and HA or FLAG tagged PPARγ2 were cloned from PSGS-PPARγ2 (Doitsh G & Shaul Y (2004) Molecular and cellular biology 24(4):1799-1808). The different mutations in PPARγ2 were generated by site-directed mutagenesis. Flag PPARγ2 constructs were cloned into pBabe puro. To generate cell lines stably expressing the different PPARγ2 constructs, retrovirus infection was performed by transfecting 293T cells with pBabe puro Flag PPARγ2 constructs or the respective empty vectors. For stable knocking down c-Abl, 293T cells were transfected with the mission pLKO.1 shRNA vectors targeting c-Abl or GFP (Sigma) together with pLP1, pLP2, and pLP-VSVG packaging plasmids (Invitrogen). Forty-eight hours after transfection, viral supernatant was filtered through a 0.45-μm filter, supplemented with 8 μg/mL polybrene, and used to infect 3T3-L1 or NIH-3T3 cells. Twenty-four hours after infection, cells were selected with 2 μg/mL puromycin (Sigma) in the culture medium. All transfections were done by the calcium phosphate method. Total RNA was extracted using the PerfectPure RNA cultured cell kit (5prime) and then reverse-transcribed by iScript cDNA synthesis kit (BioRad). Quantitative RT-PCR was performed with SYBR® Green PCR Master Mix (Kapa Biosystems) using the LightCycler® 480 Instrument (Roche Diagnostics). Sequences of the oligos used in this study are listed in Table 1.

TABLE 1

| | | |
|---|---|---|
| aP2 | Fw | ATCAGCGTAAATGGGGATTTGG-SEQ ID NO: 1 |
| | Rev | GTCTGCGGTGATTTCATCGAA-SEQ ID NO: 2 |
| LPL | Fw | ATGGATGGACGGTAACGGGAA-SEQ ID NO: 3 |
| | Rev | CCCGATACAACCAGTCTACTACA-SEQ ID NO: 4 |
| PPARγ1 | Fw | GCTGAGAAGTCACGTTCTGACA-SEQ ID NO: 5 |
| | Rev | CATCTCTGTGTCAACCATGGTA-SEQ ID NO: 6 |
| PPARγ2 | Fw | TCGCTGATGCACTGCCTATG-SEQ ID NO: 7 |
| | Rev | GAGAGGTCCACAGAGCTGATT-SEQ ID NO: 8 |

Immunoblot and coimmunoprecipitation Studies: The antibodies used were: anti-HA, monoclonal anti-β-actin, anti-Flag M2, and anti-Flag M5 (Sigma); anti c-Abl K12 and anti-general phosphotyrosine (PY20) (Santa Cruz Biotechnology); anti-E-cadherin (BD Biosciences); anti phospho-Tyr245 c-Abl (Cell Signaling). For immunoprecipitation (IP) of HA- and Flag-tagged proteins, anti-Flag M2 agarose and anti-HA agarose (Sigma) were used. Horseradish peroxidase-conjugated secondary antibodies were from Jackson Laboratories. Enhanced chemiluminescence was performed with the EZ-ECL kit (Biological Industries) and signals were detected by the ImageQuant LAS 4000 (GE Healthcare).

### Example 1

### c-Abl activation in adipogenesis

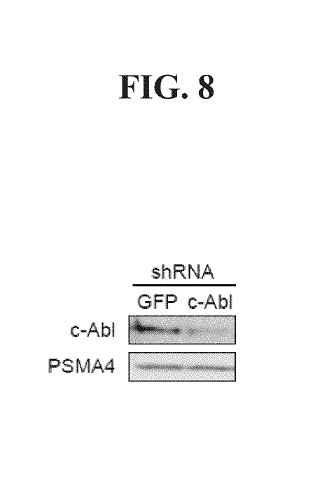

c-Abl activation was followed during adipocyte differentiation using an antibody detecting the c-Abl Y245 autophosphorylated form, a hallmark of an active kinase. c-Abl was activated on the first day of differentiation induction and remained active up to mid-differentiation stage (FIG. 1A). Next, c-Abl kinase activity was inhibited using the c-Abl inhibitor STI-571 (Imatinib). STI-571 is also an inhibitor of the tyrosine kinases c-kit and platelet derived growth factor receptor (PDGFR). While c-kit is not expressed in 3T3-L1, PDGFR is a negative regulator of adipocyte differentiation but is only expressed during the first hours of adipogenesis, after which it rapidly declines to an undetectable level. To minimize potential inhibition of PDGFR and specifically target c-Abl, STI-571 was added 7 hours after differentiation onset and treatment was maintained until day 5. Remarkably, even at low inhibitor concentration, adipogenesis was markedly reduced (FIG. 1B). Similar results were obtained when c-Abl was depleted using a specific lentiviral vector encoding shRNA (FIG. 1C and FIG. 8). Under this condition expression of the major adipogenic regulator, PPARy, and two of its important transcriptional targets, aP2 and LPL was markedly reduced (FIG. 1D). Interestingly, while PPARγ protein level was shown to decrease with c-Abl inhibition, transcription of both PPARγ isoforms was unchanged whereas transcription of their target genes was markedly reduced (FIG. 1E). These results suggest that c-Abl regulates PPARγ expression at the protein level in normal adipocyte differentiation.

### Example 2

### c-Abl regulates accumulation of PPARγ2

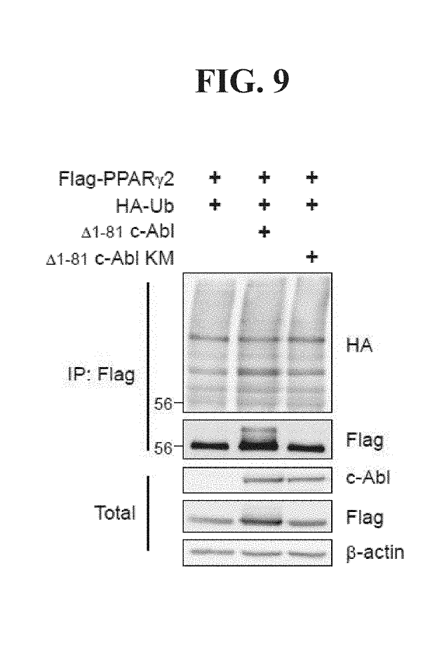

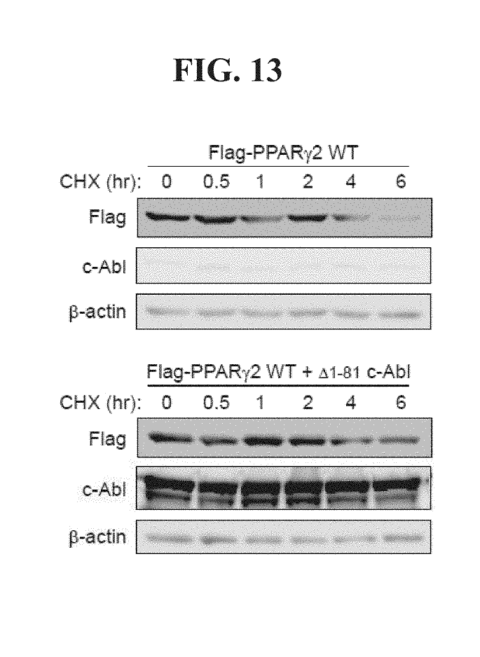

The present inventors investigated whether c-Abl might play a role in PPARγ2 accumulation. To check whether the low level of endogenous PPARγ2 expression in c-Abl depleted cells is the result of protein destabilization, they treated c-Abl-depleted 3T3-L1 cells with MG-132, a proteasomal inhibitor (FIG. 2A). PPARγ2 protein level markedly increased after proteasomal blockade, suggesting a role for c-Abl in PPARγ2 accumulation during adipocyte differentiation. Next, the present inventors measured PPARγ2 protein half-life in the presence or absence of an active form of c-Abl (41-81 c-Abl). Following translation inhibition by cycloheximide, the level of PPARγ2 gradually dropped to a minimal level within 6 hrs (FIG. 2B, see also below FIG. 13). Remarkably, in the presence of constitutively active c-Abl PPARγ2 decay was much slower. To investigate the possibility that c-Abl supports PPARγ2 stabilization via inhibition of the poly-ubiquitination of PPARγ2, an octameric tandem fusion of HA-tagged ubiquitin was co-expressed together with Flag-tagged PPARγ2 with or without active c-Abl and the pattern of the poly-ubiquitinated PPARγ2 ladder was analyzed. PPARγ2 ubiquitination was indeed significantly inhibited by c-Abl in a kinase-dependent manner (FIG. 2C and FIG. 9). These data suggest that active c-Abl promotes the stability of PPARγ2 protein.

### Example 3

### c-Abl Interacts with PPARγ2 and Phosphorylates it

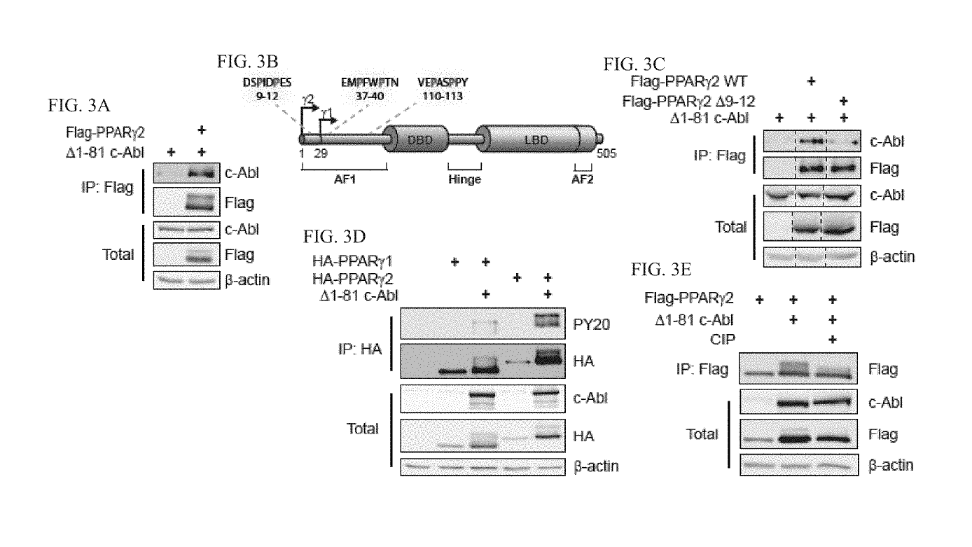

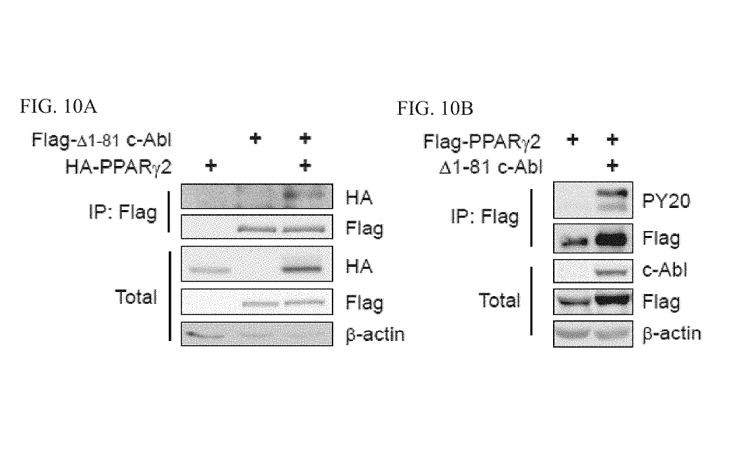

Having demonstrated that c-Abl is a positive regulator of adipogenesis and its kinase activity is essential for this role, the present inventors next asked whether PPARγ2 is a direct substrate of c-Abl. First, they measured their possible physical association in transfected HEK293 cells. When flag-tagged PPARγ2 was immunoprecipitated, a substantial amount of c-Abl was brought down as well (FIG. 3A). In a reciprocal experiment PPARγ2 was co- immunoprecipitated with c-Abl (FIG. 10A). c-Abl binds proteins through its SH3 domain to the PxxP motifs of the target protein (26). Three PxxP motifs are found in PPARγ2, all of which reside within the AF1 domain (FIG. 3B). The first, 9PxxP12, is located in the unique PPARγ2 N-terminal 30 amino acids that is absent in PPARγ1, the isoform not involved in adipogenesis (6, 27). To check whether the 9PxxP12 is involved in binding to c-Abl, a four amino acids deletion mutant of PPARγ2 was generated (49-12). Remarkably, c-Abl failed to bind this mutant (FIG. 3C).

Next the present inventors investigated PPARγ2 phosphorylation by c-Abl. They first looked for a difference in the degree of phosphorylation by c-Abl between PPARγ2, containing this motif, and the PPARγ1 isoform lacking it. Interestingly, when probing with a phosphotyrosine specific antibody it was found that unlike HA-PPARγ2, HA-PPARγ1 was only poorly phosphorylated by c-Abl (FIG. 3D). Similar results were obtained using Flag-PPARγ2 (FIG. 10B). PPARγ2 phosphorylation was accompanied by an accumulation of the PPARγ2 protein and by a substantial up-shift in the migration pattern on the gel, a fact that may suggest several phosphorylation sites. To verify this possibility, the experiment was repeated in the presence of Alkaline phosphatase. This resulted in the disappearance of the upshifted form of PPARγ2, implying that the mobility shift is the consequence of phosphorylation (FIG. 3E). These data suggest that c-Abl binds PPARγ2 through the N-terminal PxxP motif and subsequently phosphorylates it.

### Example 4

### Phosphorylation of Two Tyrosine Residues of PPARγ2 by c-Abl Increases PPARγ2 Accumulation

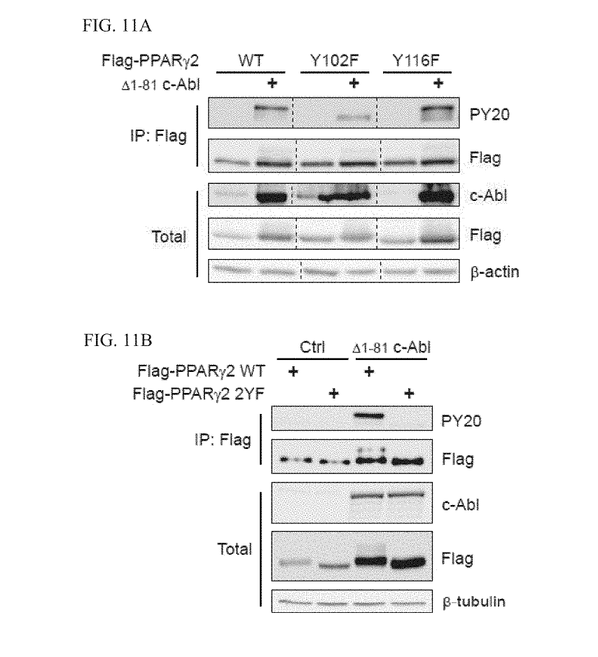

PPARγ is regulated either by ligand binding to its LBD or by other factors, mainly through its AF1 domain (FIG. 3B). To search for potential c-Abl phosphorylation sites on PPARγ2, the present inventors investigated the AF1 domain, as important modifications take place in this domain (7, 8). To this end, tyrosine residues in the AF1 domain were mutated into the phospho-dead residue phenylalanine and the PPARγ2 mutants were analyzed for phosphorylation by c-Abl. Unlike the wild type, the PPARγ2 Y78F and Y102F mutants were not upshifted in the presence of active c-Abl, but the latter displayed a certain phosphorylation level (FIG. 4A FIG. 11A). A double Y78F and Y102F mutant (PPARγ2 2YF) was not tyrosine phosphorylated in the presence of active c-Abl (FIG. 11B), delineating these two tyrosine residues as the major c-Abl targets. To check whether these tyrosine residues are phosphorylated by endogenous c-Abl during differentiation, PPARγ2 was stably expressed in NIH-3T3 mouse fibroblasts, which lack endogenous PPARγ2. These cells can be induced to differentiate into adipocytes similar to pre-adipocytes (28). PPARγ2 that was immunoprecipitated from undifferentiated NIH-3T3 cells was moderately tyrosine phosphorylated (FIG. 4B). Remarkably, at mid-differentiation PPARγ2 but not PPARγ2 2YF was markedly tyrosine phosphorylated. These data suggest that c-Abl phosphorylates PPARγ2 on tyrosine residues 78 and 102, with tyrosine 78 being the major phosphorylation site.

Next, the present inventors wished to check whether tyrosine phosphorylation by c-Abl is a mechanism by which c-Abl promotes PPARγ2 stabilization. To chemically mimic the phosphorylation state of PPARγ2 to uncouple it from the presence of c-Abl, the present inventors generated a double Y78E and Y102E mutant (PPARγ2 2YE). They then measured protein half-life of PPARγ2 in cycloheximide-treated cells. While the protein level of both the wild type PPARγ2 and the 2YF mutant gradually decreased within 4 hr, that of the phosphomimetic PPARγ2 2YE mutant remained constant (FIG. 4C). To investigate whether the increased stabilization of the 2YE mutant is the result of inhibition of its poly-ubiquitination, they analyzed the different PPARγ2 constructs when coexpressed with an HA-tagged ubiquitin. As expected, PPARγ2 ubiquitination was significantly reduced when the two c-Abl targeted tyrosines were phosphomimetically mutated, as compared to the wild type or phospho-dead mutated PPARγ2 (FIGS. 4D and 4E). These data suggest that c-Abl modifies PPARγ2 to escape degradation.

### Example 5

### PPARγ2 is Activated by c-Abl

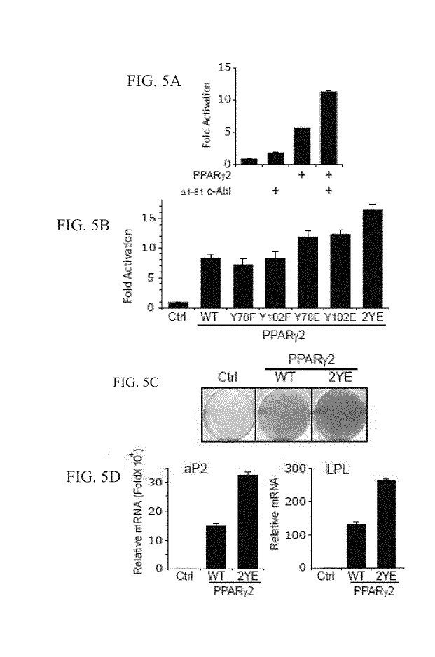

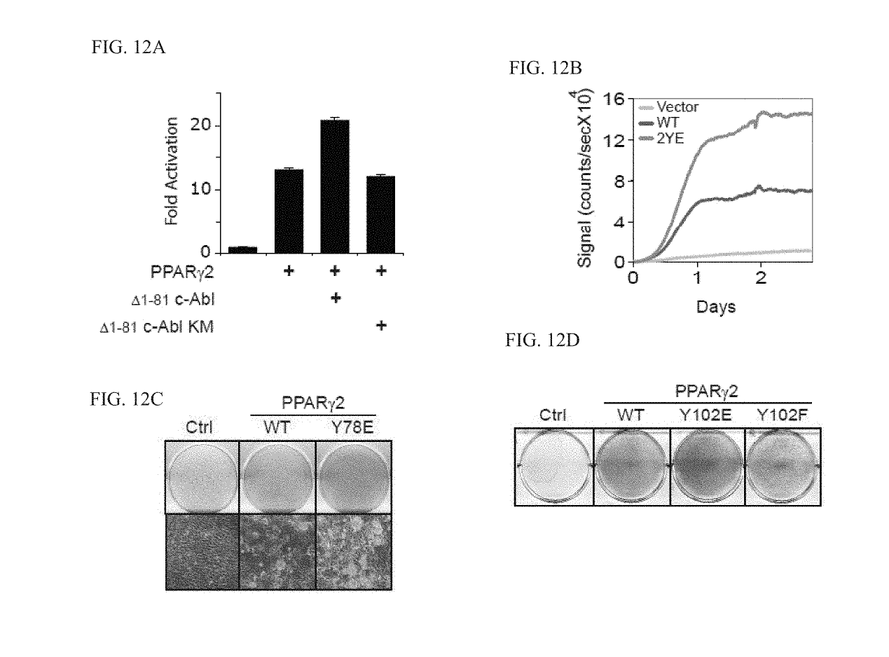

To investigate the role of c-Abl in inducing PPARγ2 activity, the PPRE3 reporter construct containing three copies of the PPARγ responsive element sequence (29) was used. In the presence of active c-Abl, PPARγ2-induced activation of the promoter was doubled as compared to induction with PPARγ2 alone (FIG. 5A). A c-Abl kinase-dead mutant failed to do so (FIG. 12A), suggesting that c-Abl in a kinase-dependent manner supports PPARγ2 transcription activity.

Next, the present inventors investigated PPARγ2 mutated at the tyrosine residues undergoing phosphorylation by c-Abl in transcription. As compared to either wild type PPARγ2 or the phospho-dead mutants, the Y78E and Y102E phosphomimetic mutants were more active (FIG. 5B). Interestingly, the PPARγ2 2YE double mutant was most active in this assay and in a real time bioluminescence recording assay (FIG. 12B). These data support a model whereby Y78 and Y102 phosphorylation potentiate PPARγ2 activity.

The present inventors next asked whether these PPARγ2 tyrosine residues are involved in adipocyte differentiation. To this end, they overexpressed either wild type PPARγ2 or the phosphomimetic 2YE mutant in NIH-3T3 mouse fibroblasts. The wild type PPARγ2 cells displayed a moderate adipogenic phenotype as assessed by oil red staining (FIG. 5C). Remarkably, overexpression of the PPARγ2 2YE phosphomimetic mutant resulted in a substantial increase in differentiation level, both at the level of oil red staining (FIG. 5C) and the PPARγ target genes aP2 and LPL (FIG. 5D). The single phosphomimetic mutants were also more active than the wild type (FIGS. 12C-D). These data suggest that tyrosine phosphorylation of PPARγ2 at the residues targeted by c-Abl play a role in adipogenesis.

### Example 6

### c-Abl Binding Site on PPARγ2 is a Functional Genetic Polymorphism Site

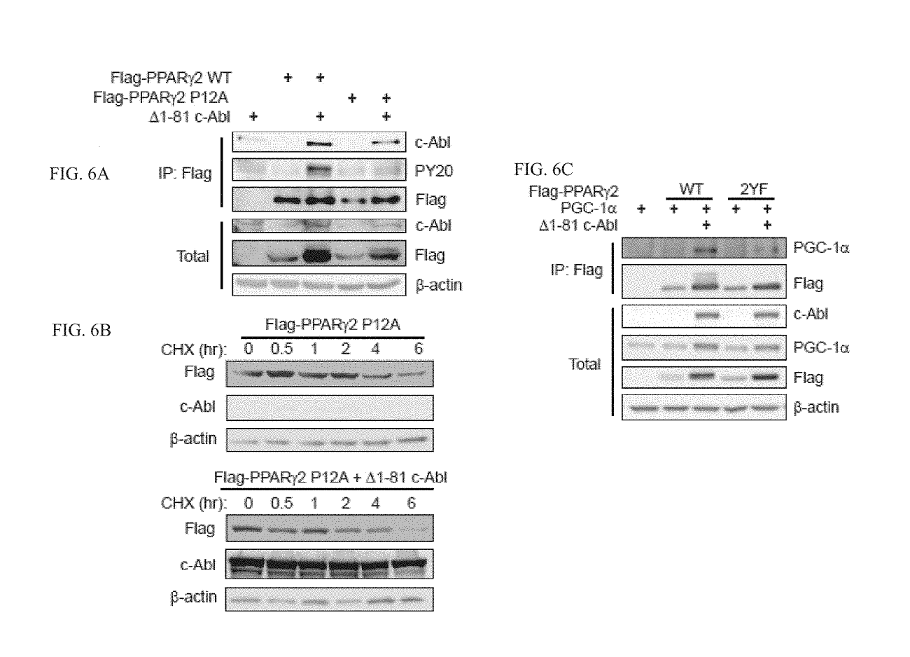

As mentioned above, c-Abl was found to bind PPARγ2 through the N-terminal 9PxxP12 sequence. Interestingly, P12 (Pro12) is polymorphic and some individuals carry alanine at this position, the common Prol2Ala variant (9). The present inventors therefore wished to check whether polymorphism influences regulation of PPARγ2 by c-Abl. Remarkably, the P12A variant was inefficient in binding to c-Abl and in undergoing phosphorylation by the kinase (FIG. 6A). Furthermore, this variant did not accumulate with the expression of active c-Abl (FIG. 6B and FIG. 13).

In a recent work, the Prol2Ala variant was found to poorly bind coactivators like PGC-1α (11), which is an important component in metabolism regulation by PPARγ (30). To check if c-Abl may affect interaction of PPARγ2 with PGC-1α, both proteins were expressed in HEK293 cells and PGC-1α was co-immunoprecipitated in the presence or absence of active c-Abl. While c-Abl enhanced binding of PGC-1α to wild type PPARγ2, the phospho-dead mutant 2YF was significantly less efficient in PGC-1α binding in the presence of c-Abl (FIG. 6C). Since the expression level of the wild-type and mutant PPARγ in this experiment was the same, this suggests that tyrosine phosphorylation of PPARγ plays a more direct role in regulating interaction with co-factors, in addition to the effect on PPARγ stability. These data suggest that reduced affinity of the PPARγ2 P12A variant to its coactivators may be explained by impairment in c-Abl binding and tyrosine phosphorylation.

### Example 7

The checkpoint kinase 1 (Chk1) regulates among DNA damage response (DDR). Chk1 inhibitors such as UCN-01 have been developed and demonstrated to have antitumor activity.

Recruitment/activation of ATM/ATR and “sensor” proteins recruits Chk1 at damage sites, where the latter are activated. ATR (predominantly) or ATM (to a lesser extent) phosphorylates Chk1 at Ser317/345, directly leading to activation. Since c-Abl (a component of DDR) is activated in the process of adipogenesis the present inventors asked whether Chk1 is activated as well.

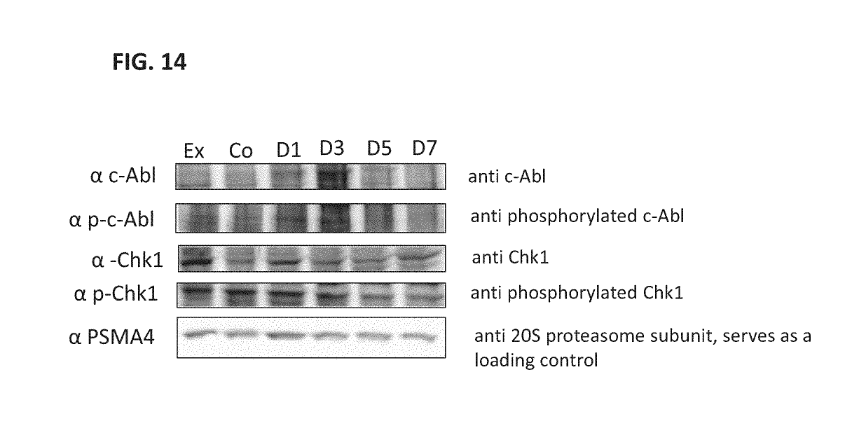

Chk1 activation was followed during 3T3L1 differentiation to adipocytes. It was found to remain active during this process (FIG. 14).

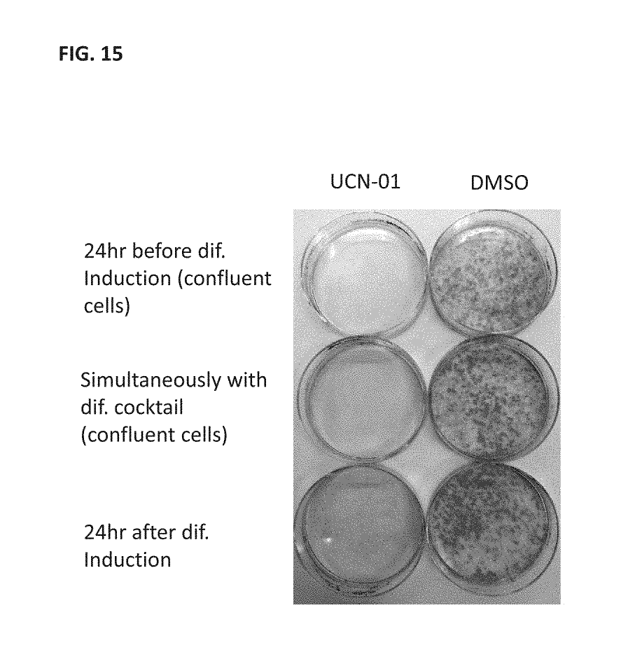

UCN-01, a known inhibitor of Chk1 was found to completely inhibit the differentiation process (FIG. 15).

### REFERENCES

1. Rosen E D (2002) The molecular control of adipogenesis, with special reference to lymphatic pathology. Annals of the New York Academy of Sciences 979:143-158; discussion 188-196.
2. Castillo G, et al. (1999) An adipogenic cofactor bound by the differentiation domain of PPARgamma. The EMBO journal 18(13):3676-3687.
3. Bugge A, Grontved L, Aagaard M M, Borup R, & Mandrup S (2009) The PPARgamma2 A/B-domain plays a gene-specific role in transactivation and cofactor recruitment. Molecular endocrinology (Baltimore, Md 23(6):794-808.
4. Shao D, et al. (1998) Interdomain communication regulating ligand binding by PPAR-gamma. Nature 396(6709):377-380.
5. Chambon P (2005) The nuclear receptor superfamily: a personal retrospect on the first two decades. Molecular endocrinology (Baltimore, Md 19(6):1418-1428.
6. Tontonoz P, Hu E, Graves R A, Budavari A I, & Spiegelman B M (1994) mPPAR gamma 2: tissue-specific regulator of an adipocyte enhancer. Genes & development 8(10): 1224-1234.
7. Ohshima T, Koga H, & Shimotohno K (2004) Transcriptional activity of peroxisome proliferator-activated receptor gamma is modulated by SUMO-1 modification. The Journal of biological chemistry 279(28):29551-29557.
8. Hu E, Kim J B, Sarraf P, & Spiegelman B M (1996) Inhibition of adipogenesis through MAP kinase-mediated phosphorylation of PPARgamma. Science (New York, N.Y 274(5295):2100-2103.
9. Yen C J, et al. (1997) Molecular scanning of the human peroxisome proliferator activated receptor gamma (hPPAR gamma) gene in diabetic Caucasians: identification of a Prol2Ala PPAR gamma 2 missense mutation. Biochemical and biophysical research communications 241(2):270-274.
10. Deeb S S, et al. (1998) A Prol2Ala substitution in PPARgamma2 associated with decreased receptor activity, lower body mass index and improved insulin sensitivity. Nature genetics 20(3):284-287.
11. Heikkinen S, et al. (2009) The Prol2Ala PPARgamma2 variant determines metabolism at the gene-environment interface. Cell metabolism 9(1):88-98.
12. Yuan ZM, et al. (1996) Role for c-Abl tyrosine kinase in growth arrest response to DNA damage. Nature 382(6588):272-274.
13. Agami R, Blandino G, Oren M, & Shaul Y (1999) Interaction of c-Abl and p73alpha and their collaboration to induce apoptosis. Nature 399(6738):809-813.
14. Levy D, Adamovich Y, Reuven N, & Shaul Y (2008) Yapl phosphorylation by c-Abl is a critical step in selective activation of proapoptotic genes in response to DNA damage. Molecular cell 29(3):350-361.
15. Bennett R L & Hoffmann F M (1992) Increased levels of the Drosophila Abelson tyrosine kinase in nerves and muscles: subcellular localization and mutant phenotypes imply a role in cell-cell interactions. Development (Cambridge, England) 116(4):953-966.
16. Daniel R, Wong P M, & Chung S W (1996) Isoform-specific functions of c-abl: type I is necessary for differentiation, and type IV is inhibitory to apoptosis. Cell Growth Differ 7(9):1141-1148.
17. Koch A, et al. (2000) Direct interaction of nerve growth factor receptor, TrkA, with non-receptor tyrosine kinase, c-Abl, through the activation loop. FEBS letters 469(1):72-76.
18. Lee Y C, et al. (2010) Src family kinase/abl inhibitor dasatinib suppresses proliferation and enhances differentiation of osteoblasts. Oncogene 29(22):3196-3207.
19. O'Neill A J, Cotter T G, Russell J M, & Gaffney E F (1997) Abl expression in human fetal and adult tissues, tumours, and tumour microvessels. The Journal of pathology 183(3):325-329.
20. Frasca F, et al. (2007) Role of c-Abl in directing metabolic versus mitogenic effects in insulin receptor signaling. The Journal of biological chemistry 282(36):26077-26088.
21. Wilson B, Liotta LA, & Petricoiniii E (2013) Dynamic protein pathway activation mapping of adipose-derived stem cell differentiation implicates novel regulators of adipocyte differentiation. Molecular & cellular proteomics: MCP 12(9):2522-2535.
22. Brasher BB & Van Etten RA (2000) c-Abl has high intrinsic tyrosine kinase activity that is stimulated by mutation of the Src homology 3 domain and by autophosphorylation at two distinct regulatory tyrosines. The Journal of biological chemistry 275(45):35631-35637.
23. Buchdunger E, et al. (2000) Abl protein-tyrosine kinase inhibitor STI571 inhibits in vitro signal transduction mediated by c-kit and platelet-derived growth factor receptors. The Journal of pharmacology and experimental therapeutics 295(1): 139-145.
24. Matsubara Y, Suzuki H, Ikeda Y, & Murata M (2010) Generation of megakaryocytes and platelets from preadipocyte cell line 3T3-L1, but not the parent cell line 3T3, in vitro. Biochemical and biophysical research communications 402(4):796-800.
25. Vaziri C & Faller D V (1996) Down-regulation of platelet-derived growth factor receptor expression during terminal differentiation of 3T3-L1 pre-adipocyte fibroblasts. The Journal of biological chemistry 271(23):13642-13648.
26. Ren R, Mayer B J, Cicchetti P, & Baltimore D (1993) Identification of a ten-amino acid proline-rich SH3 binding site. Science (New York, N.Y 259(5098):1157-1161.
27. Ren D, Collingwood T N, Rebar E J, Wolffe A P, & Camp H S (2002) PPARgamma knockdown by engineered transcription factors: exogenous PPARgamma2 but not PPARgammal reactivates adipogenesis. Genes & development 16(1):27-32.
28. Tontonoz P, Hu E, & Spiegelman BM (1994) Stimulation of adipogenesis in fibroblasts by PPAR gamma 2, a lipid-activated transcription factor. Cell 79(7):1147-1156.
29. Zhang B, et al. (1992) Identification of a peroxisome proliferator-responsive element upstream of the gene encoding rat peroxisomal enoyl-CoA hydratase/3-hydroxyacyl-CoA dehydrogenase. Proceedings of the National Academy of Sciences of the United States of America 89(16):7541-7545.
30. Puigserver P, et al. (1998) A cold-inducible coactivator of nuclear receptors linked to adaptive thermogenesis. Cell 92(6):829-839.
31. Tsai K K & Yuan Z M (2003) c-Abl stabilizes p73 by a phosphorylation-augmented interaction. Cancer research 63(12):3418-3424.
32. Shaul Y (2000) c-Abl: activation and nuclear targets. Cell death and differentiation 7(1):10-16.
33. Baskaran R, et al. (1997) Ataxia telangiectasia mutant protein activates c-Abl tyrosine kinase in response to ionizing radiation. Nature 387(6632):516-519.
34. Yang D Q & Kastan M B (2000) Participation of ATM in insulin signalling through phosphorylation of eIF-4E-binding protein 1. Nature cell biology 2(12):893-898.
35. Altanerova V, Horvathova E, Matuskova M, Kucerova L, & Altaner C (2009) Genotoxic damage of human adipose-tissue derived mesenchymal stem cells triggers their terminal differentiation. Neoplasma 56(6):542-547.
36. Lee H, Lee Y J, Choi H, Ko E H, & Kim J W (2009) Reactive oxygen species facilitate adipocyte differentiation by accelerating mitotic clonal expansion. The Journal of biological chemistry 284(16): 10601-10609.
37. Tybulewicz V L, Crawford C E, Jackson P K, Bronson R T, & Mulligan R C (1991) Neonatal lethality and lymphopenia in mice with a homozygous disruption of the c-abl proto-oncogene. Cell 65(7):1153-1163.
38. Fitter S, et al. (2008) Long-term imatinib therapy promotes bone formation in CML patients. Blood 111 (5):2538-2547.
39. Fitter S, et al. (2010) Plasma adiponectin levels are markedly elevated in imatinib-treated chronic myeloid leukemia (CML) patients: a mechanism for improved insulin sensitivity in type 2 diabetic CML patients? The Journal of clinical endocrinology and metabolism 95(8):3763-3767.
40. Fitter S, Vandyke K, Gronthos S, & Zannettino A C (2012) Suppression of PDGF-induced PI3 kinase activity by imatinib promotes adipogenesis and adiponectin secretion. Journal of molecular endocrinology 48(3):229-240.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In the claims:

1. A method of treating obesity in a subject in need thereof comprising administering to the subject a therapeutically effective amount of an agent that down-regulates an amount and/or activity of Abelson murine leukemia viral oncogene homolog 1 (c-Abl) or checkpoint kinase 1 (Chk1).

2. A method of reducing the amount of fat content or body weight of a subject comprising administering to the subject a therapeutically effective amount of an agent that down-regulates an amount and/or activity of Abelson murine leukemia viral oncogene homolog 1 (c-Abl) or checkpoint kinase 1 (Chk1).

3. (canceled)

4. The method of claim 1, wherein said agent is a small molecule inhibitor.

5. The method of claim 1, wherein said agent is a polynucleotide agent directed against said c-Abl or said Chk1.

6. The method of claim 4, wherein said agent is selected from the group consisting of imatinib (STI-571), nilotinib, bosutinib, INNO-406, MK-0457 and PD173955.

7. The method of claim 4, wherein said agent is UCN-01 or LY2606368.

8. The method of claim 1, wherein said agent is not co-administered with an anti-cancer agent or an agent which is used to treat atherosclerosis.

9. (canceled)

10. The method of claim 1, wherein the subject has the amino acid proline at position 12 of Peroxisome proliferator-activator receptor gamma (PPARγ2).

11. The method of claim 1, wherein the subject does not have cancer.

12. The method of claim 1, wherein the subject does not have diabetes.

13. The method of claim 1, wherein the subject does not have pathological lipid levels.

14. The method of claim 1, wherein the subject does not have atherosclerosis.

15. The method of claim 1, wherein the subject does not have pathological cholesterol levels.

16. The method of claim 1, wherein the subject has been treated for at least one month to correct for elevated lipid levels.

17-19. (canceled)

20. The method of claim 2, wherein said agent is a small molecule inhibitor.

21. The method of claim 20, wherein said agent is selected from the group consisting of imatinib (STI-571), nilotinib, bosutinib, INNO-406, MK-0457 and PD173955.

22. The method of claim 20, wherein said agent is UCN-01 or LY2606368.

23. The method of claim 1, wherein the subject does not have atherosclerosis, pathological lipid levels or diabetes.

24. The method of claim 2, wherein the subject does not have atherosclerosis, pathological lipid levels or diabetes.
